(19) United States
(12) Patent Application Publication
(10) Pub. No.: US 2023/0222659 A1
(43) Pub. Date: Jul. 13, 2023

(54) CONJOINED TWIN NETWORK FOR TREATMENT AND ANALYSIS

(71) Applicant: University of Connecticut, Farmington, CT (US)

(72) Inventors: Sheida Nabavi, Wellesley, MA (US); Clifford Yang, Farmington, CT (US); Jun Bai, Vernon, CT (US)

(21) Appl. No.: 18/096,700

(22) Filed: Jan. 13, 2023

Related U.S. Application Data
(60) Provisional application No. 63/299,313, filed on Jan. 13, 2022.

(51) Int. Cl. G06T 7/00; A61B 6/00

(52) U.S. Cl. CPC G06T 7/0014; A61B 6/502; G06T 2207/30068; G06T 2207/30096; G06T 2207/20084; G06T 2207/20081

(57) ABSTRACT

A method includes receiving first data based on a region of interest of tissue. The first data may be captured to represent the tissue according to a first moment. The method also includes receiving second data based on the region of interest. The second data may be captured to represent the tissue according to a second moment different from the first moment. The method also includes determining features of the first data according to a first network. The first network may comprise weights. The method also includes determining features of the second data according to the weights. The method also includes determining an input based on the features of the first data and the features of the second data. The method may also include treating a patient or adjusting treatment of the patient diagnosed by one or more of these steps. An apparatus for performing the method is disclosed.

### CROSS-REFERENCE TO RELATED APPLICATIONS

This application claims the benefit of U.S. Provisional Application No. 63/299,313, filed Jan. 13, 2022, which is incorporated herein by reference in its entirety.

### BACKGROUND

Early detection and treatment of abnormal tissues can lead to positive outcomes in treatment and survival. For example, abnormal tissue may be indicative of breast and other cancers. Breast cancer is the most common cancer in women and is also the leading cause of death for women between the ages of 20 and 59. Screenings for breast cancer and other abnormal tissues have provided chronological documentation of tissue growth and development. Computer-aided detection reduces the risk of overlooking growth, but the over-detection and under-detection provided by these methods can increase the recall rate when used to interpret mammograms and other data, causing misdiagnosis and costs to rise.

### SUMMARY

Methods, apparatuses, systems, and techniques are described for treatment and analysis of patients. For a better understanding of the underlying concepts, there follows specific non-limiting examples:

A method includes receiving first data based on a region of interest of tissue. The first data may be captured to represent the tissue according to a first moment. The method also includes receiving second data based on the region of interest. The second data may be captured to represent the tissue according to a second moment different from the first moment. The method also includes determining features of the first data according to a first network. The first network may comprise weights. The method also includes determining features of the second data according to the weights. The method also includes determining an input based on the features of the first data and the features of the second data. The method also includes determining an abnormality in the tissue according to an application of the input on a second network. The method may also include treating a patient or adjusting treatment of the patient diagnosed by one or more of these steps.

An apparatus includes one or more processor. The apparatus includes one or more non-transitory computer-readable medium. The one or more non-transitory computer-readable medium includes a first network having weights and a second network configured to output an indication of an abnormality. The input of the second network may be based on an output of the first network. The one or more non-transitory computer-readable medium includes instructions operable upon execution by the one or more processor to receive first data based on a region of interest of tissue. The first data may be captured to represent the tissue according to a first moment. The instructions are further operable upon execution by the one or more processor to receive second data based on the region of interest. The second data may be captured to represent the tissue according to a second moment different from the first moment. The instructions are further operable upon execution by the one or more processor to determine features of the first data according to the first network and the weights. The instructions are further operable upon execution by the one or more processor to determine features of the second data according to the weights. The instructions are further operable upon execution by the one or more processor to determine the input based on the features of the first data and the features of the second data. The instructions are further operable upon execution by the one or more processor to determine an abnormality in the tissue according to an application of the input on a second network.

A method includes receiving first data based on a region of interest of tissue. The first data may be captured to represent the tissue according to a first moment. The method includes treating or adjusting treatment to a patient associated with the tissue. The patient may be diagnosed by a process that includes receiving second data based on the region of interest. The second data may be captured to represent the tissue according to a second moment different from the first moment. The process may include determining features of the first data according to a first network. The first network may include weights. The process may include determining features of the second data according to the weights. The process may include determining an input based on the features of the first data and the features of the second data. The process may include determining an abnormality in the tissue according to an application of the input on a second network.

### BRIEF DESCRIPTION OF THE DRAWINGS

In order to provide understanding techniques described, the figures provide non-limiting examples in accordance with one or more implementations of the present disclosure, in which:

FIG. 1 illustrates an example system for treating a patient with abnormal tissue;

FIG. 2 illustrates example data based on tissue;

FIG. 3 illustrates an example conjoined twin network;

FIG. 4 illustrates an example method for determining an abnormality;

FIG. 5 illustrates a method for training one or more networks; and

FIG. 6 illustrates an example network architecture

FIG. 7 illustrates example results.

### DETAILED DESCRIPTION

Full-field digital mammography (FFDM) scans are among the most challenging medical images for automatic cancer classification, due to the characteristics of breast tissues. The heterogeneous tree-shaped structure of the breast has a connected tissue network that supports glandular tissues. These breast tissues are also surrounded by fat and covered with skin. Thus, a breast tumor can be occult because of overlaying glandular architecture. In addition, some breast tumors show identical characteristics of glandular tissues. Cancer may be identified based on the features extracted from individual breast exams. As discussed, some breast tumors look similar to breast normal tissues, making the classification of objects and abnormal tissues challenging.

Detection of abnormal tissue can be achieved with higher levels of accuracy than previously attained by using a conjoined twin network that fuses features determined based on neural networks (e.g., convolutional neural networks) to compare data (e.g., images) from previous screenings to data from contemporaneous screenings to identify changes in tissue that may be abnormal. The data may be used as paired inputs to predict the probability of malignancy. One or more distance learning functions may be employed to compare features detected within the data. The architecture may be configured to receive high-dimensional input for detection of very small malignancies in dense breasts (e.g., microcalcifications, occult tumors). For example, the architecture of one or more of the neural networks and distance learning functions discussed herein constitute a technical improvement to the art not previously realized. The architecture disclosed herein provides enhanced treatment options and treatment accuracy for patients to reduce the risk of overlooking growth and reduce the over-detection and under-detection of such growths, reducing misdiagnosis and the over-treatment or under-treatment of disease. The present disclosure at least presents improvements to machine learning architectures and the technical field of tumor treatment.

In order to provide some context, aspects of certain terms are presented. As used herein, the term “weights” generally references to the real values that are associated with each input/feature and they convey the importance of that corresponding feature in predicting the final output. Features with weights that are close to zero said to have lesser importance in the prediction process compared to the features with weights having a larger value. “Inputs” generally refers to a set of values for which an output value will be predicted or estimated. Inputs can be viewed as features or attributes in a dataset.

Networks may be employed to detect interclass and intraclass features. For example, two parallel networks may have the same or similar weights. The weights may be trained by a one-shot learning algorithm. A distance learning network may be used to compare the outputs from the respective networks. For example, the distance learning network may measure the distance between the feature maps from each of the networks and then applies a fully connected or dense layer to learn the differences between the feature maps (e.g., interclass features). The parallel network may have an architecture based on a residual network (e.g., RESNET). A distance learning network may be based on a correlation matrix that compares current and previous images. For example, an N×N symmetric correlation matrix C in RN×N, where N is the size of the feature vectors and employs a shallow CNN to generate similarity feature vector. A loss function may include Barlow loss. The Barlow loss may act as a regularizer or normalizer. For example, the loss function (e.g., the function that determines model performance, or portion thereof) may be based on a Barlow loss function described in Equations 1 and 2 below.

Bₗₒₛₛ [symbol] Σᵢ(1−Cᵢᵢ)²+λΣᵢΣ_{j≠i}Cᵢⱼ² (1)

where λ is a predetermined quantity (e.g., a positive constant) that trades off between Σᵢ(1−Cᵢᵢ)² and ΣᵢΣ_{j≠i}Cᵢᵢ², and where C is the cross-correlation matrix computed between outputs of the networks (e.g., networks 350, 370) along the batch dimension: e.g.,

$${C}_{ij} \frac{{\sum }_{b} {z}_{b , i}^{A} {z}_{b , j}^{B}}{\sqrt{{\sum }_{b} {( {z}_{b , i}^{A} )}^{2}} \sqrt{{\sum }_{b} {( {z}_{b , j}^{B} )}^{2}}} \qquad ( 2 )$$

where b indexes batch samples and i, j index the vector dimension based on the networks (e.g., networks 350, 370). For example, the vector dimension may be based on one or more outputs of the networks. C is a square matrix sized with a dimensionality based on the networks (e.g., networks 350, 370). For example, C may be based on one or more outputs of the networks. The C matrix may be comprised of values between negative one and positive one. Normalization may transform network information (e.g., input information) to a predetermined scale (e.g., between 0 and 1). Regularization may transform weights, through training and the loss function, to improve performance (e.g., reduce over-fitting).

The feature representations may allow comparisons of the data using one or more distance functions. For example, the distance function may measure the similarity between the two functions.

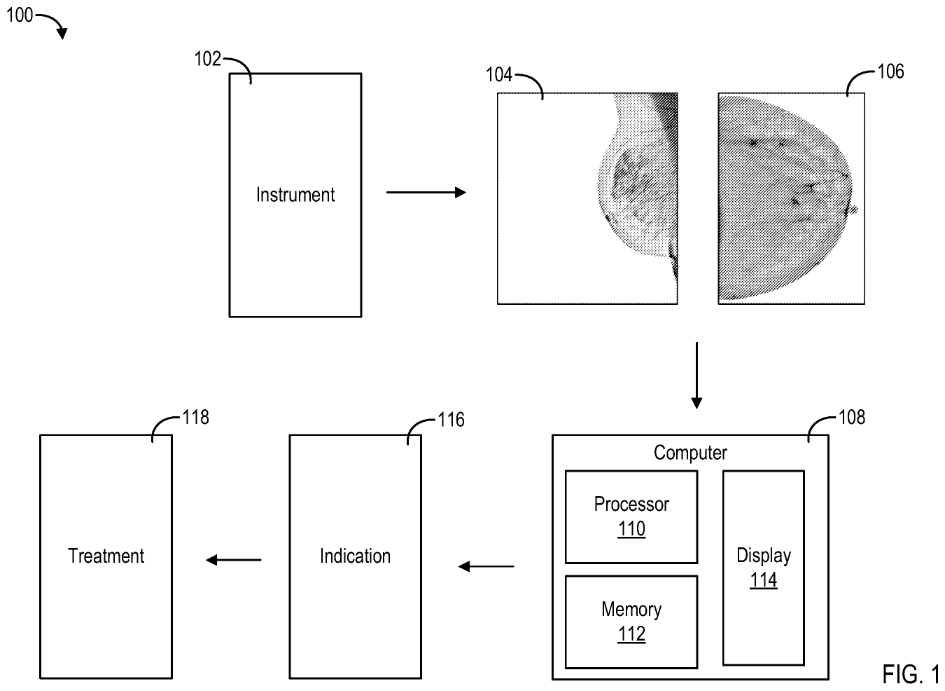

Referring to FIG. 1, an example system 100 for treating a patient with abnormal tissue in accordance with one or more implementations of the present disclosure is shown. The system 100 includes an instrument 102 for determining data associated with a patient. The instrument 102 may be an apparatus configured to collect tissue, electromagnetic waves, fluids, or other sensory information related to the patient. For example, the instrument may collect reflected or undisturbed X-rays, ultrasound waves, visual light waves, or other electromagnetic waves to provide data 104, 106 regarding tissue or other bodily components based on the patent. For the example shown, the instrument is configured to collect X-ray, computed tomography (CT), or magnetic-resonance images (MRI) from the patient to generate data 104, 106.

The data 104, 106 may be represented in various dimensions. For example, the data 104, 106 may be one-dimensional, two-dimensional, three-dimensional, multidimensional, or various combinations thereof. As shown, the data 104, 106 is a two-dimensional image representative of a breasts or mammary glands. For example, the data 104, 106 may be provided by the instrument as a pixel or voxel representation of the tissue. The data 104, 106 may further include metadata or relational data derived from the tissue, the instrument, or otherwise.

The data 104, 106 may be provided to a computer 108. The instrument 102 and the computer 108 may be unitary, sharing the same housing, or in communication with one another over a network or communications bus. For example, the instrument 102 may be configured to send the data 104, 106 to a repository. The repository may be in the cloud or otherwise situated. The repository may be configured to store and maintain numerous data sets from multiple patients. The computer 108 may be configured to access the repository over a network on demand. The data sets may be accessed for training or inference. For example, the computer 108 may be used to train a network stored within the memory 112 of the computer 108. The memory 112 may include various computer-readable storage mediums as discussed herein. A processor 110 or a combination of processors 110 may be used to conduct processing on the data 104, 106 and define a network stored within the memory 112. The processor 110 may be a combination of various processing types for generally processing and machine learning. For example, the processor 110 may include application specific integrated circuits (ASIC), field-programmable gate arrays (FPGA), graphics processing units, central processing units, or combinations thereof. The processing of data may be distributed across various chasses and infrastructure. For example, the processing may be conducted in the cloud over multiple instances, containers, repositories, or combinations thereof. The networks and data may be stored over multiple instances, containers, repositories or combinations thereof.

The computer 108 may include a display 114 for providing an indication 116 of data categorization. For example, the display 114 may display a category of the data 104, 106 based on a network stored within the memory 112. The display 114 may be located with the computer 108 or near a patient room or instrument room.

The indication 116 may be categorical (e.g., normal, abnormal, unknown), probabilistic (e.g., 25% probability of abnormality), or otherwise. The indication 116 may be provided to a repository or online medical system. For instance, the indication 116 may be communicated to a patient, doctor, or other medical personnel through an online portal. Medical personnel may apply or adjust treatment 118 based on the indication. For example, an indication 116 suggesting that the tissue is abnormal would compel medical personnel to perform surgery, chemotherapy, hormonal therapy, immunotherapy, or radiation therapy, additional testing, or a combination of surgery, chemotherapy, hormonal therapy, immunotherapy, radiation therapy, or additional testing. The dosage of certain therapies may be automatically or manually applied or adjusted based on the indication 116. For example, the quantity or periodicity of chemotherapy or other therapies may be adjusted based on the indication 116. The screening periodicity may be adjusted based on the indication 116, adjusting or reducing medical costs. For example, the indication 116 may present a low probability of abnormality, requiring additional screen in one year instead of six months. Other applications or adjustments are contemplated.

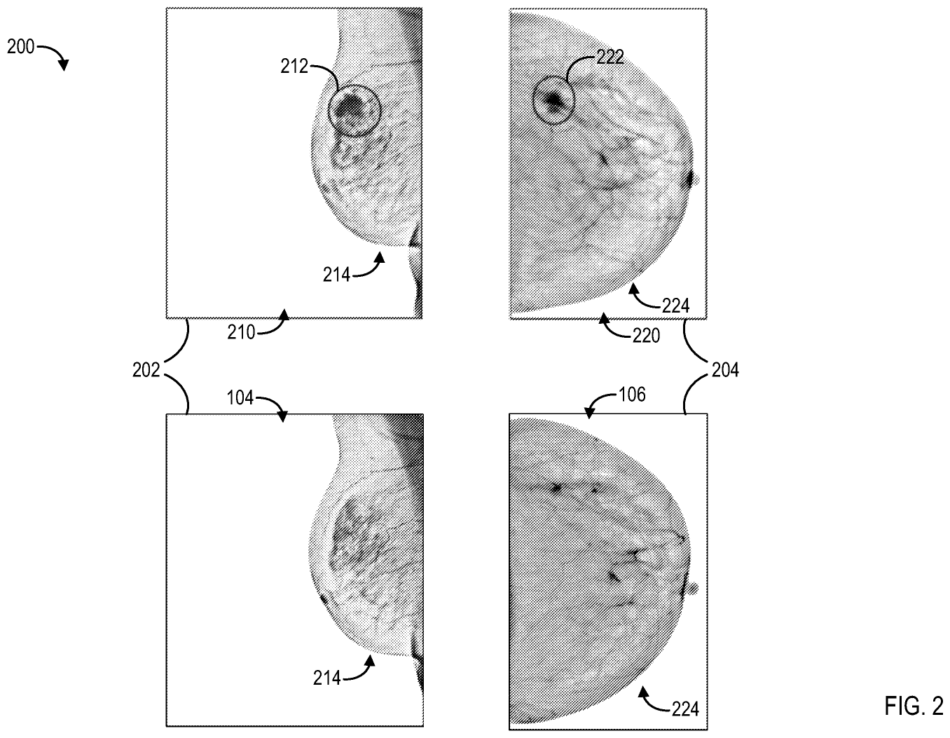

In FIG. 2, example data 200 based on tissue in accordance with one or more implementations of the present disclosure is shown. For example, data 104, 106 may be based on a region of interest 202, 204 for two different patients having respective tissues. The region of interest 202, 204 may be a portion of the data captured or all of the data captured by the instrument 102. For example, the region of interest 202, 204 may be based on an aspect of the instrument 102. The data 104, 106 may be captured according to a first moment. For example, breast mammograms may be captured using FFDM.

The patient may be screened annually or otherwise for abnormalities within the breast tissue. In this way, the data 104, 106 may be captured according to a first moment. The first moment may be a specific day or time when the data 104, 106 is captured according to the screening schedule. The data 104, 106 may be defined based on when the complete set of data is stored in a repository, an average time that the data was taken or otherwise. For example, the data may be captured over a week and assigned a moment pertaining to the time that the data 104, 106 is stored within the repository. The data 210, 220 may be captured according to a second moment. For example, the data 210, 220 may be captured a year, or about a year after the first moment. Other screening periods are contemplated (e.g., hourly, daily, biannually). The data 210, 220 may be captured from the same aspect with the same region of interest 202, 204 to maintain the continuity of the data 104, 106 captured according to the first moment with data 210, 220 captured according to the second moment. The data 104, 106 from the first moment may be compared with data 210, 220 from the second moment, indicating an abnormality of tissues 214, 224 of different patients, respectively.

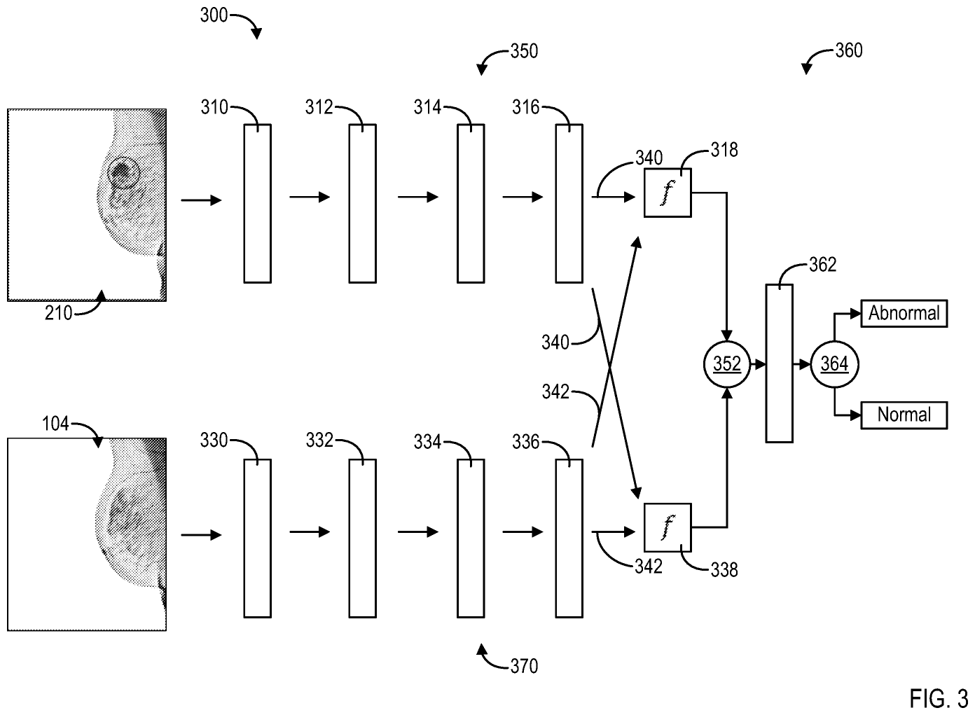

In FIG. 3, an example conjoined twin network 300 in accordance with one or more implementations of the present disclosure is shown. The networks shown may be stored on the memory 112 or one or more other computer-readable medium. A network 350 may be configured to receive data. For example, the network 350 may receive data 104 based on the first moment and data 210 based on the second moment. For example, the network 350 may receive the data 104 based on the first moment and data 210 based on the second moment, the data 104, 210 being obtained at different points in time from the same region of interest 202, 204 of patent tissue 214.

The network 350 may receive data 210 with the first layer 310. The network 350 may have the same weights, or substantially similar weights, as the network 370. For example, the first layer 310 of network 350 may have substantially similar weights to the first layer 330 of network 370. Substantially similar weights may be indicated where the weights are identical or based on a pre-trained network with one-shot training or application specific training. For instance, fine-tuning may change all or some of the weights. As data 210 and data 104 pass through the first layers 310, 330 of respective networks 350, 370 they are subjected to the same weights. As such, similar features are extracted from the data 104, 210 by respective layers 330, 310. The features extracted from data 210 are passed through layers 310, 312, 314 of network 350 to extract features. The layers 310, 312, 314 may have substantially similar weights of respective layers 330, 332, 334 of network 370. Various quantities or types (e.g., convolutional, pooling, fully connected) of layers may be used by the respective networks 350, 370.

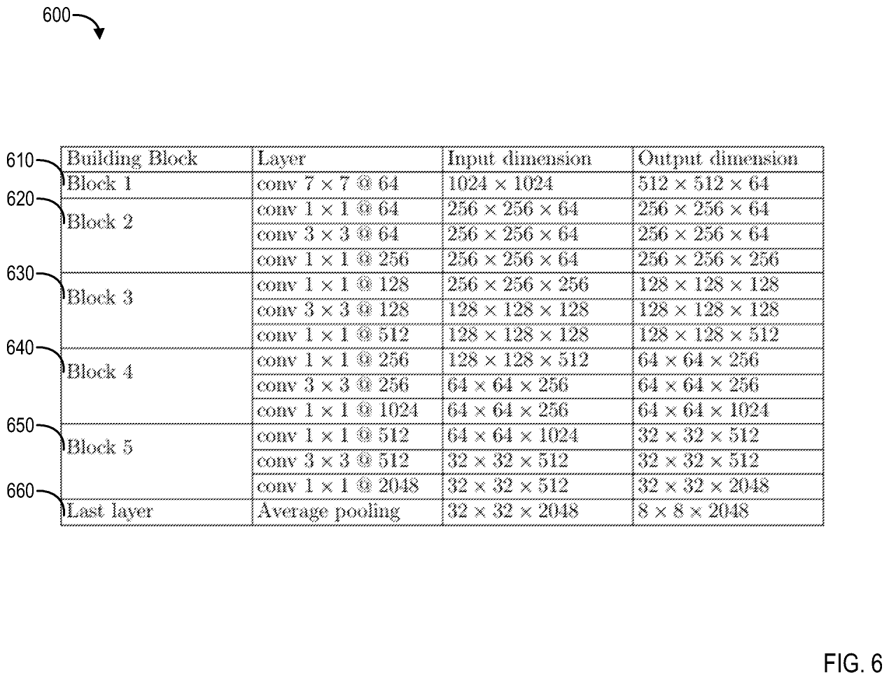

For example, FIG. 6 depicts example convolutional layers (e.g., blocks) for one of the networks 350, 370. The layers 310, 312, 314 of network 350 may culminate in a pooling layer 316 (e.g., average pooling layer) of network 350. The layers 330, 332, 334 of network 370 may culminate in a pooling layer 336 (e.g., average pooling layer) of network 370. The resulting features 340, 342 of the respective pooling layers 316, 336 are then used to form an input 352 to network 360. The network 360 may be a fully connected network that learns the differences between the features maps that would indicate abnormal tissues from the data 104, 210. For example, a distance network may be used to quantify or determine the differences between features 340, 342 generated based on networks 350, 370 having substantially similar weights and original data 104, 210. Features 340, f_{c}, (e.g., features from contemporaneous data) defined by pooling layer 316 and features 342, fₚ, (e.g., features from previously gathered data) defined by pooling layer 336 are compared to define tissue categories (e.g., normal, abnormal, unknown), probabilities (e.g., 25% probability of abnormality), etc. Features 340, 342 may be flatted feature maps or feature vectors of the respective data 104, 210. The features may be used as inputs in distance learning functions 318, 338. For example, distance learning function 318 may be based on Equation 3.

dᵢ=f_{c}−fₚ (3)

where d₁ measures the pixel-wise distance (e.g., component distance) of f_{c} and fₚ. Distance learning function 338 may be based on Equation 4.

d₂=\sqrt{Σⱼ₌₀^{m}f_{c}^{j}−fₚ^{j}} (4)

where d₂ measures the scalar, Euclidean distance of f_{c} and fₚ, and m is the size of the feature vectors. A concatenation block may operate as an input 352 to network 360, where d₁ is concatenated with d₂ to build the distance feature for determination of abnormal tissue. The network 360 may include any number of layers 362. The layers 362 may output to a sigmoid function, as provided in Equation 5, that predicts the probability of dissimilarity (e.g., abnormal) or similarity (e.g., normal).

ŷ=sigmoid(w^{T}[d₁ [symbol] d₂]+b (5)

where w denotes the vector of weights, b denotes bias, [symbol] denotes concatenation, and ŷ represents the predicted probability of similarity. In such a way, the conjoined twin network can output the likelihood of abnormal changes between current year and previous year images. Binary cross-entropy may be used as a loss function to train the network.

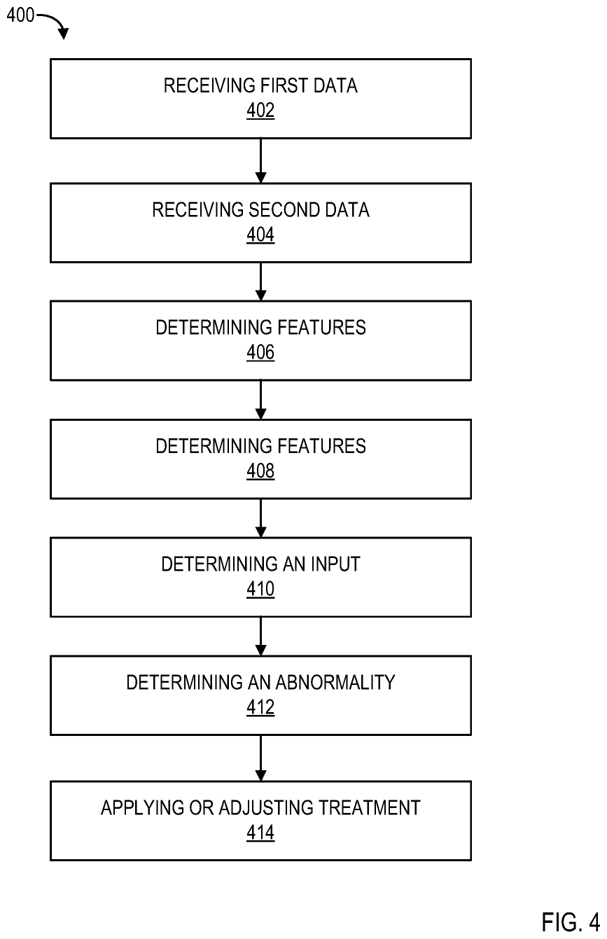

In FIG. 4, an example method 400 for determining an abnormality in accordance with one or more implementations of the present disclosure. The steps presented herein may be performed in whole or in part by any of the components described herein. Any of the steps may be omitted, duplicated, or rearranged. For example, in step 402 data may be received. The data may be data 104 that is captured according to a first moment. In step 404, data may be received. The data may be data 210 that is captured according to a second moment. The first moment may be different from the second moment. For example, the second moment may be after the first moment.

In step 406, features 340 may be determined according to a network 350 based on the data 210. The network 350 may include weights. The weights may be the same as the weights of network 370. In step 408, the features 342 of the data 104 may be determined according to the same weights as network 350. The features 342 may be determined by the network 350 or the network 370. In step 410, an input 352 (e.g., concatenation block) may be determined based on the features 340, 342. The input may be based on one or more distances determined between the features 340, 342. For example, the distance may be a pixel-wise distance. The pixel-wise distance may be based on a difference between a vector representation (e.g., series of component values) of the features 340 and features 342. The distance may also be a scalar. The scalar distance may be determine based on a Euclidean distance between features 340 and features 342. The input may be based on both distances or additional distances (e.g., a correlation or covariance matrix). For instance, the input may be a concatenation of multiple distances flattened for input into network 360. In step 412 an abnormality of the tissue 214 may be determined based on the input and network 360.

In step 414, a treatment may be applied or adjusted to a patient. The treatment may be surgery, chemotherapy, hormonal therapy, immunotherapy, or radiation therapy, or a combination of surgery, chemotherapy, hormonal therapy, immunotherapy, radiation therapy. The treatment may be applied or adjusted based on the abnormality.

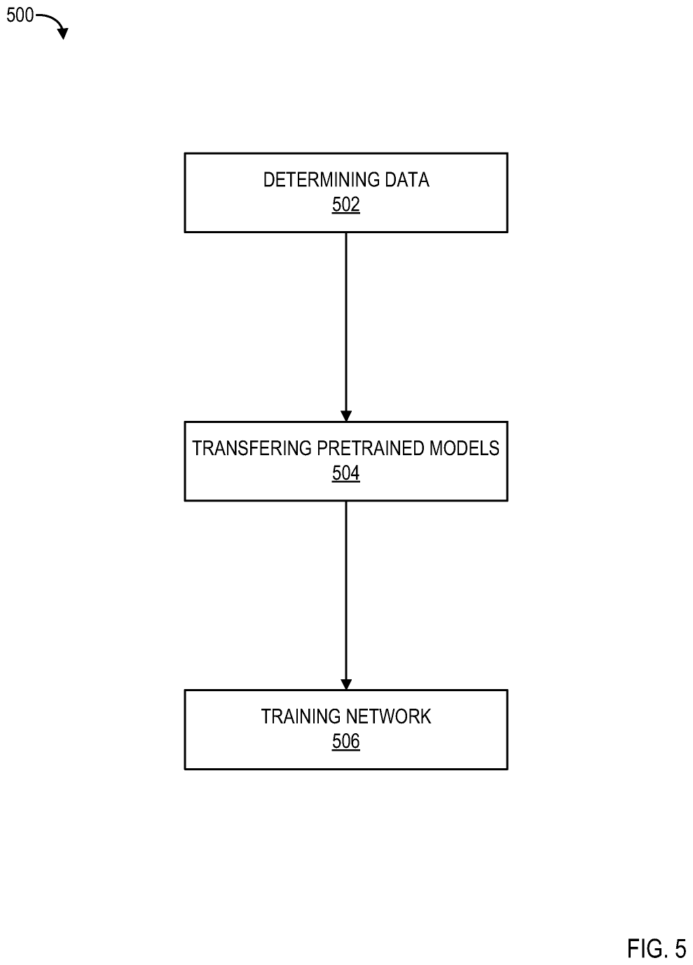

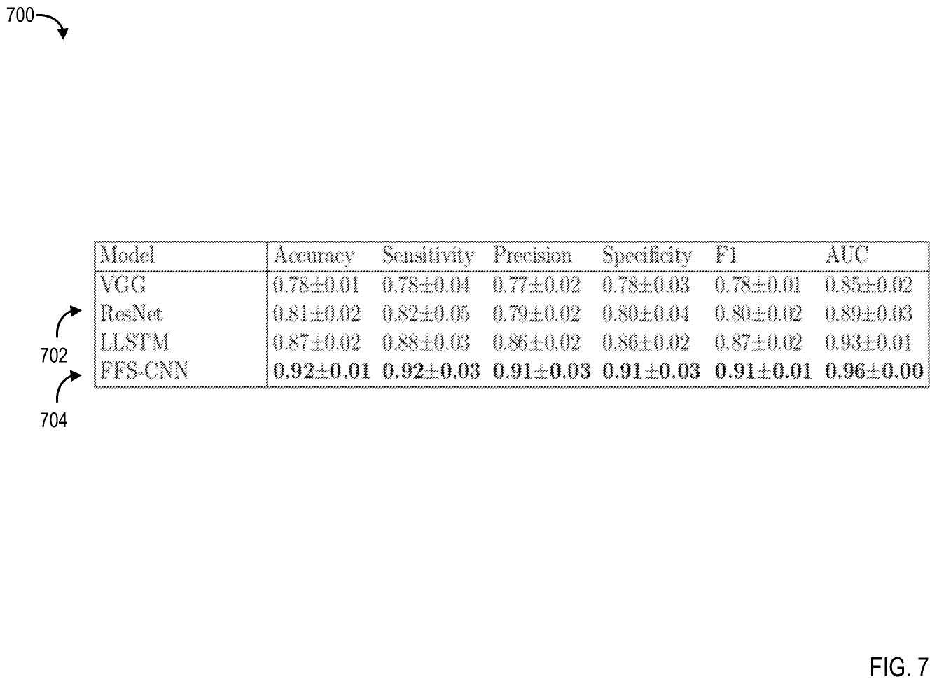

Referring to FIG. 5, a method 500 for training one or more networks in accordance with one or more implementations of the present disclosure is shown. For example, the training data may comprise curated data from one or more studies. In step 502, the test and training data may be determined. In an example, the training data may comprise only curated data or only a portion of the curated data. For example, the training data may include thousands of images from FFDM exams. For each patient, images may be collected from previous year and current year FFDM exams. The images may be labeled for classifying abnormal and normal tissue. For training networks each image may be paired with its corresponding previous year image and each image with its corresponding previous year image (left/right breast, CC/MLO view). To reduce the unnecessary computational cost, the black background may be removed from the original FFDM images as much as possible. An algorithm may be used to detect the widest breast from the data set and set the cutting margin as 20 pixels away from the widest breast skin edge. In addition, all annotations and metal marks may be removed from all the FFDM images. To increase the size of the training data set, data augmentation may be used. For example, rotation (e.g., 90, 180, and 270 degrees) and Contrast Limited Adaptive Histogram Equalization (CLAHE). In step 504, the networks 350, 370 may be pre-trained ResNet networks with pretrained weights to initialize the backbone networks for all the networks. Weights for other networks (e.g., network 360) may be randomly assigned or assigned with a normal distribution (e.g., Xavier). The pretrained weights may be unfrozen during the training process of step 506. As such, the weights of networks 350, 370 may adjusted slightly and differ from one another. Dropout may be used to prevent overfitting. In addition, an L1 regularizer (e.g., 1e.5 to 2) and L2 regularizer (e.g., 1e.4 to 2) may be used in fully-connected layers. Networks trained and implemented in such a new and different way are beyond what is achievable by pen and paper or prior techniques, removing or reducing the time-consuming and laborious—and quite often inaccurate—behavior of manual analysis. Further, techniques described herein are not those previously used in a manual process. These specific techniques, as described herein, for training and application of networks are an improvement in technology or technical field that at least includes one or more of artificial intelligence, radiography or other imaging techniques, and oncology. As shown in FIG. 7, the techniques described herein at least improve the treatment of disease by ensuring the proper level of treatment is administered. Further, the techniques described herein do not pre-empt every method of improving treatment or monopolize the basic tools of scientific or technological work.

In FIG. 6, an example network architecture 600 in accordance with one or more implementations of the present disclosure is shown. The example network architecture may be used in networks 350, 370, for example. The network architecture contains five building blocks 610, 620, 630, 640, 650 with respective layers followed with an average pooling layer 660. The size of the layers, kernels, and hyper parameters are for example only. In the first building block 610, there is a 7×7 convolutional layer with a batch normalization layer and the ReLU activation layer. Max pooling is also applied after the first building block. The other building blocks 620, 630, 640, 650 contain convolutional blocks and identity blocks. Each convolutional block and identity block may have three convolutional layers, three batch normalization layers and three activation layers. The kernel size may be 1×1, 3×3, or otherwise. The purpose of convolutional blocks is to reduce feature dimensions, therefore, a 1×1 convolutional layer and batch normalization layer is added to the short cut path of the convolutional blocks 610, 620, 630, 640, 650. To adjust for two classes (single neuron output), the top layers of ResNet and are removed and two fully connected layers are added, with dimensions of 512, and 256 with an output layer. A ReLU activation function for the fully connected layers (e.g., 362). The output may be a single neuron, and a sigmoid function 364 may be applied to obtain the likelihood of abnormal and normal.

In FIG. 7, example results 700 are shown in accordance with one or more implementations of the present disclosure. The results 700 indicate the performance of one or more techniques described herein indicated as FFS-CNN 704. The results 700 also include other techniques 702 and the lower performance associated with such techniques. For example, one or more of the techniques described herein resulted in higher sensitivity and specificity in determining abnormal tissues than before, which may provide for improved treatments of abnormal tissues. Further, the accuracy and precision is also improved through one or more of the techniques described herein, as indicated.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a special purpose computer or other programmable data processing instrument to produce a machine, such that the processor-executable instructions which execute on the computer or other programmable data processing instrument create a device for implementing the functions specified in the flowchart block or blocks.

These processor-executable instructions may also be stored in a computer-readable memory or a computer-readable medium that may direct a computer or other programmable data processing instrument to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing instrument to cause a series of operational steps to be performed on the computer or other programmable instrument to produce a computer-implemented process such that the processor-executable instructions that execute on the computer or other programmable instrument provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for using a machine learning classifier(s) for detection and classification. Machine learning (ML) is a subfield of computer science that gives computers the ability to learn through training without being explicitly programmed. Machine learning methods include, but are not limited to, deep-learning techniques, naïve Bayes classifiers, support vector machines, decision trees, neural networks, and the like.

The method steps recited throughout this disclosure may be combined, omitted, rearranged, or otherwise reorganized with any of the figures presented herein and are not intend to be limited to the four corners of each sheet presented.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
administering or adjusting treatment to a patient diagnosed according to a process comprising:
receiving first data based on a region of interest of tissue of the patient, the first data captured to represent the tissue according to a first moment;
receiving second data based on the region of interest, the second data captured to represent the tissue according to a second moment different from the first moment;
determining features of the first data according to a first network, the first network having weights;
determining features of the second data according to the weights;
determining an input based on the features of the first data and the features of the second data; and
determining an abnormality in the tissue according to an application of the input on a second network;
wherein the treatment comprises surgery, chemotherapy, hormonal therapy, immunotherapy, or radiation therapy, or a combination of surgery, chemotherapy, hormonal therapy, immunotherapy, radiation therapy.

2. The method of claim 1, wherein determining an input comprises:
determining a distance between the features of the first data and the features of the second data.

3. The method of claim 2, wherein the distance is a pixel-wise distance and determining a distance comprises:
determining a difference between a vector representation of the features of the first data and a vector representation of the features of the second data.

4. The method of claim 2, wherein the distance is scalar and determining the distance comprises:
determining a Euclidean distance between a vector representation of the features of the first data and a vector representation of the features of the second data.

5. The method of claim 1, wherein the determining the input further comprises:
determining a difference between a vector representation of the features of the first data and a vector representation of the features of the second data; and
determining a Euclidean distance between the vector representation of the features of the first data and the vector representation of the features of the second data.

6. The method of claim 5, wherein the determining the input further comprises:
concatenating the difference and the Euclidean distance.

7. The method of claim 1, wherein the second network comprises a sigmoid function configured to distinguish the abnormality from normality.

8. The method of claim 1, wherein the second moment is before the first moment based on a screening period.

9. The method of claim 1, wherein the weights are trained by one-shot learning.

10. The method of claim 1, wherein determining the features of the second data according to the weights is further based on a third network comprising the weights.

11. An apparatus comprising:
at least one processor; and
one or more non-transitory computer-readable medium comprising:
a first network having weights and a second network configured to output an indication of an abnormality, wherein an input of the second network is based on an output of the first network; and
instructions operable upon execution by the at least one processor to:
receive first data based on a region of interest of tissue, the first data captured to represent the tissue according to a first moment;
receive second data based on the region of interest, the second data captured to represent the tissue according to a second moment different from the first moment;
determine features of the first data according to the first network and the weights;
determine features of the second data according to the weights;
determine the input based on the features of the first data and the features of the second data; and
determine the abnormality in the tissue according to an application of the input on the second network.

12. The apparatus of claim 11, further comprising:
a display configured to indicate the abnormality.

13. The apparatus of claim 11, wherein the instructions for the determination of the input are further operable upon execution by the at least one processor to:
determine a distance between the features of the first data and the features of the second data.

14. The apparatus of claim 13, wherein the distance is a pixel-wise distance and the instructions for the determination of the distance are further operable upon execution by the at least one processor to:
determine a difference between a vector representation of the features of the first data and a vector representation of the features of the second data.

15. The apparatus of claim 14, wherein the distance is scalar and the instructions for the determination of the distance are further operable upon execution by the at least one processor to:
determine a Euclidean distance between a vector representation of the features of the first data and a vector representation of the features of the second data.

16. The apparatus of claim 11, wherein the instructions for the determination of the input are further operable upon execution by the at least one processor to:
determine a difference between a vector representation of the features of the first data and a vector representation of the features of the second data; and
determine a Euclidean distance between the vector representation of the features of the first data and the vector representation of the features of the second data.

17. The apparatus of claim 16, wherein the instructions for the determination of the input are further operable upon execution by the at least one processor to:
concatenate the difference and the Euclidean distance.

18. A method comprising:
receiving first data based on a region of interest of tissue of a patient, the first data captured to represent the tissue according to a first moment;
receiving second data based on the region of interest, the second data captured to represent the tissue according to a second moment different from the first moment;
determining features of the first data according to a first network, the first network comprising weights;
determining features of the second data according to the weights;
determining an input based on the features of the first data and the features of the second data; and
determining an abnormality in the tissue according to an application of the input on a second network.

19. The method of claim 18, wherein the determining the input further comprises:
determining a difference between a vector representation of the features of the first data and a vector representation of the features of the second data; and
determining a Euclidean distance between the vector representation of the features of the first data and the vector representation of the features of the second data.

20. The method of claim 19, wherein the determining the input further comprises:
concatenating the difference and the Euclidean distance.
